(19) United States
(12) Patent Application Publication
(10) Pub. No.: US 2003/0224385 A1
(43) Pub. Date: Dec. 4, 2003

(54) Targeted genetic risk-stratification using microarrays

(76) Inventor: German Pihan, Weston, MA (US)

Correspondence Address: J. PETER FASSE, Fish & Richardson P.C., 225 Franklin Street, Boston, MA, 02110-2804, US

(21) Appl. No.: 10/313,211

(22) Filed: Dec. 6, 2002

Related U.S. Application Data
(60) Provisional application No. 60/338,442, filed on Dec. 7, 2001.
(60) Provisional application No. 60/423,793, filed on Nov. 5, 2002.

(51) Int. Cl. C12Q 1/68; C12P 19/34

(52) U.S. Cl. 435/6; 435/91.2

(57) ABSTRACT

The invention relates to new expedient and cost-effective assays that are capable of identifying many or all relevant diagnostic and prognostic genetic lesions in cancer or cancer predisposition using multiplex PCR or other nucleic acid amplification or enrichment technology in conjunction with bead microarrays for the purpose of risk-stratifying patients with cancer or cancer predisposition. The new assay methods are referred to herein as BARCODE-MT for Bead ARray COded DEtection of Multiple Targets. These assays are high-throughput, and can be automated for highly accurate diagnoses that can be used to optimize risk-adapted therapy.

### CROSS REFERENCE TO RELATED APPLICATIONS

This application claims priority from U.S. Provisional Patent Application Serial No. 60/338,442 filed Dec. 7, 2001, and U.S. Provisional Patent Application Serial No. 60/423,793 filed Nov. 5, 2002, which are incorporated herein by reference in their entireties.

### TECHNICAL FIELD

This invention relates to assays for diagnostic, prognostic and therapeutic risk-stratification in cancer and cancer predisposition.

### BACKGROUND

Monitoring and testing for genetic changes are both informative and important to the study of disease (e.g., cancer) and to clinical practice (e.g., cancer diagnosis and treatment). A large and diverse group of genetic changes, including but nor limited to, genetic point mutations, genomic imbalances such as chromosomal rearrangements or fluctuations in gene copy number, polymorphisms, gene expression differences, and the presence of viral or bacterial nucleic acid in a subject carry important risk-stratifying information. The detection and analysis of these changes are important in the molecular classification of disease, and are useful in providing differential diagnosis of disease, confirmation of disease, risk of adverse prognosis and in pharmacogenomic applications such as in the tailoring of treatment to the individual. Specifically, it allows the optimization of therapy for an individual based upon the particular genetic signature or genetic expression profile of a tumor. Genetic signatures are a set of differences in genes that can classify an individual as having a specific disease, being at risk for contracting a specific disease, or as being one of a group of individuals representing a subgroup within a disease. For example, a genetic “signature” of malignant melanoma may help explain how malignant melanoma can spread to other parts of the body. Using gene expression profiling, researchers have found a genetic signature, or set of differences in genes, that can be used to divide patients with advanced melanoma into subgroups. Such classification of cancer offers the possibility of more accurately determining the prognosis of a particular patient's tumor, based on his or her genetic makeup, and offers the hope of tailoring therapies to the individual.

Genetic signatures can also be used to monitor qualitative and quantitative aspects of treatment efficacy, resistance mechanisms, and can uncover novel gene targets for cancer-specific therapies. They are also useful in comparing gene expression in the same sample under different environmental conditions, for characterizing therapeutic agents, and for recognizing potential therapy toxicity. Transcriptional profiling on solid phase microarrays is becoming a powerful technique for the molecular classification of cancer (see, e.g., Ferrando et al., Cancer Cell, 1:75-87 (2002); Singh D et al., Cancer Cell, 1:203-9 (2002); Pomeroy et al., Nature, 415:436-42 (2002); Ship et al., Nature Medicine, 8:68-74 (2002); and Armstrong et al., Nat Genet 30:41-7 (2002)).

Transcriptional profiling involves the simultaneous quantification of tens of thousands of mRNAs by hybridization of the whole transcriptome in the form of cRNA to cDNA or oligonucleotide probes arrayed on solid supports. Through the use of image analysis and statistical algorithms (e.g., hierarchical clustering) “expression profile signatures” can be extracted from these comprehensive analyses. Diagnostic, prognostic, and therapeutic response signatures can be derived that can then be used to interrogate newly diagnosed tumors. Transcriptome-wide expression profiling assays, however, can be complex, costly, and time consuming.

The use of targeted genomic or transcriptional profiling of specific diseases is more manageable than genome and transcriptome-wide profiling assays. Such analysis can allow the determination of individual differences, e.g., genetic signatures, for a specific disease. Various disorders are associated with genetic lesions that can be used to establish a diagnosis, or determine a patient's prognosis, or potential response to a particular therapy. Such disorders include acute myeloid and lymphoid leukemia, chronic lymphoid and myeloproliferative disorders, lymphomas, sarcomas and epithelial cancers such as lung, breast, prostate, cervical, anogenital, and colon carcinomas. Because current therapies for these disorders can have significant risks and side-effects, it is important to tailor or optimize therapy according to risk-factors for a particular disorder.

For example, acute leukemia is clinically and biologically heterogeneous. Nevertheless, certain defined genetic lesions within this large group of hematopoietic stem cell neoplasms, mostly in the form of chromosome translocations, are known to predict, better than any other clinicopathologic feature, clinical behavior and/or response to therapy. In the last few years, it has become clear that the residual clinical heterogeneity among leukemia with risk-defining translocations, and that of leukemia without translocations, is due to the presence of additional genetic lesions that may exert combinatorial effects on prognosis. See, e.g., Willman, Semin. Hematol., 36:390-400 (1999). Ongoing microarray mRNA expression analysis holds promise in defining relatively homogeneous groups of leukemia that are likely to share biological and genetic features. It is almost certain that multiple genetic lesions in leukemia are the rule rather than the exception (see, e.g., Dash et al., Baillieres Best Pract. Res. Clin. Hematol., 14:49-62, 2001), and that they may exert combinatorial influences on leukemia biology, prognosis, and response to therapy. Because current therapies for acute leukemia have certain associated risks, it is important to tailor therapy according to risk-factors. See, e.g., Willman, Leukemia, 15:690-4 (2001).

### SUMMARY

The invention is based on the discovery that risk stratification (e.g., risk-adapted therapy) for various disorders, such as cancer as well as conditions that predispose to cancer (e.g., viral infections), can be achieved by using an expedient and cost-effective assay that is capable of identifying many or all relevant genetic lesions using multiplex PCR and bead arrays or microarrays to identify multiple risk-stratifying genetic lesions related to specific disorders. The new assay method is referred to herein as BARCODE-MT for Bead ARray COded DEtection of Multiple Targets.

In general, the invention features a method of detecting the presence of multiple target nucleic acid molecules in a biological sample by (a) isolating and enriching (e.g., amplifying (e.g., by PCR) and/or affinity isolating) nucleic acid molecules from the biological sample; (b) treating the enriched (e.g., amplified (e.g., by PCR) and/or affinity isolated) target nucleic acid molecules with Exonuclease I; (c) performing linear PCR on the Exonuclease I treated enriched target nucleic acid molecule to produce linear PCR product; wherein only a single primer is used; (d) obtaining beads coupled to an oligonucleotide molecule complementary to the amplified target nucleic acid molecules; (e) forming a mixture by mixing the beads and the enriched linear PCR product nucleic acid; (f) forming a reacted sample by incubating the mixture under conditions wherein if the enriched linear PCR product includes the target nucleic acid molecule, the enriched linear PCR product will hybridize to the oligonucleotide molecule; (g) analyzing the reacted sample by determining the fluorescence of each bead analyzed (e.g., by detecting fluorescence, e.g., by flow cytometry); and (h) detecting the level, e.g., relative level, of fluorescence on the beads, wherein the level of fluorescence corresponds to the level, e.g., relative level, of target nucleic acid molecule in the biological sample.

The target nucleic acid molecule can be a risk-stratifying lesion, e.g., in a gene associated with cancer, e.g., leukemia, acute myelogenous leukemia, chronic myeloproliferative disorders, chronic lymphoproliferative disorders, lymphomas (Hodgkin's and non-Hodgkins), carcinomas, lung cancer, prostate cancer, breast cancer, cervical cancer, anogenital cancer, and colon cancer, and the amplification can be done by RT-PCR, PCR, RNA polymerase transcription, or some other nucleic acid amplification technology such as rolling circle amplification. Specifically, the amplification can include logarithmic PCR (or other methods), then a step in which Exonuclease I treatment is used, followed by a round of linear PCR (referred to herein as LogLinear PCR).

In addition, the method can be used to optimize risk-adapted therapy for a disorder such as leukemia associated with the target nucleic acid. In another embodiment, the nucleic acid can be RNA or DNA. In yet another embodiment, the method can be used to optimize risk-adapted therapy for a disorder (e.g., cancer, e.g., leukemia, acute myelogenous leukemia, chronic myeloproliferative disorders, chronic lymphoproliferative disorders, lymphomas (Hodgkin's and non-Hodgkins), carcinomas, lung cancer, prostate cancer, breast cancer, cervical cancer, anogenital cancer, and colon cancer) associated with the target nucleic acid molecule. In another embodiment, the method can further include the step of determining if a gene rearrangement is present or absent in the target nucleic acid molecule, determining gene dosage of the target nucleic acid molecule, and determining if the target nucleic acid molecule is mutated (e.g., a gene fusion, a gene inversion, a gene deletion, or a gene insertion), wherein presence or absence of mutation or aberrant gene dosage provides information to provide optimized diagnosis, prognosis and/or therapy.

In another aspect, the invention features a method of simultaneously detecting the presence of multiple target nucleic acid molecules in a biological sample by (a) enriching (e.g., amplifying (e.g., by PCR or RT-PCR) or affinity isolating) isolated nucleic acid from the biological sample, wherein enrichment incorporates a detectable label onto a PCR product, wherein the PCR product may comprise a target nucleic acid; (b) treating the enriched nucleic acid with Exonuclease I; (c) performing linear PCR on the Exonuclease I treated amplified nucleic acid to produce linear PCR product; wherein only a single primer is used; (d) obtaining addressable beads coupled to at least one oligonucleotide molecule complementary to the target nucleic acid; (e) mixing the addressable beads and the linear PCR product to form a mixture; (f) incubating the mixture under conditions allowing the linear PCR product to hybridize to oligonucleotide molecules that contain the target nucleic acid; (g) analyzing the incubated mixture by determine the address of each bead analyzed by its fluorescence; and (h) detecting the level, e.g., presence, absence, or relative level, of the detectable label on each of the addressable beads (e.g., by detecting fluorescence, e.g., by flow cytometry), wherein the level, e.g., relative level, of the detectable label corresponds to the level, e.g., presence, absence, or relative level, of the target nucleic acid in the biological sample.

The target nucleic acid can be a risk-stratifying lesion, e.g., in a gene associated with cancer, e.g., leukemia, and the amplification can be done by RT-PCR PCR, RNA polymerase transcription or some other nucleic acid amplification technology such as rolling circle amplification. Specifically, the amplification includes, e.g., logarithmic PCR, then a step of Exonuclease I treatment followed by a round of linear PCR (referred to herein as LogLinear PCR). Optionally, unhybridized PCR product can be removed from the incubated mixture prior to analyzing the incubated mixture. In addition, the method can be used to optimize risk-adapted therapy for a disorder such as leukemia associated with the target nucleic acid. In another embodiment, the isolated nucleic acid can consist of RNA or DNA (e.g., HPV DNA). In another embodiment the target nucleic acid is a genetic risk-stratifying lesion (e.g., associated with cancer, e.g. leukemia, acute myelogenous leukemia, chronic myeloproliferative disorders, chronic lymphoproliferative disorders, lymphomas (Hodgkin's and non-Hodgkins), carcinomas, lung cancer, prostate cancer, breast cancer, cervical cancer, anogenital cancer, and colon cancer), which can be used to optimize risk adapted therapy. In another embodiment, the detectable label (e.g., biotin) emits fluorescence. In another embodiment, the method is used to optimize risk-adapted therapy for a disorder (e.g., cancer, e.g. leukemia, acute myelogenous leukemia, chronic mycloproliferative disorders, chronic lymphoproliferative disorders, lymphomas (Hodgkin's and non-Hodgkins), carcinomas, lung cancer, prostate cancer, breast cancer, cervical cancer, anogenital cancer, and colon cancer), associated with the target nucleic acid molecule.

In another embodiment, the method can further include the step of determining if a gene rearrangement is present or absent in the target nucleic acid molecule, determining gene dosage of the target nucleic acid molecule, and determining if the target nucleic acid molecule is mutated (e.g., a gene fusion, a gene inversion, a gene deletion, or a gene insertion), wherein presence or absence of mutation or aberrant gene dosage provides information to provide optimized diagnosis, prognosis and/or therapy.

When a nucleic acid is “enriched” it is enhanced whether by being isolated or purified (e.g., made more detectable over background), or amplified (e.g., increased in number). For example, a nucleic acid can be enriched by affinity isolation or purification (e.g., affinity isolation with an antibody or affinity purification with a nucleic acid probe (e.g., a DNA probe)) or amplified by standard amplification procedures such as PCR or RT-PCR. A nucleic acid can also be enriched by alternative amplification means such as rolling circle amplification or RNA polymerase mediated linear amplification.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The invention provides a simple, fast, and cost-effective high throughput diagnostic or prognostic method, capable of detecting in a single reaction multiple risk stratifying genetic lesions and their variants in as little as six hours. The results can be used for patient risk-stratification or to provide optimized risk-adapted therapy.

Other features and advantages of the invention will be apparent from the following detailed description, and from the claims.

### DESCRIPTION OF DRAWINGS

FIGS. 1A to 1E are schematic representations of the multiplex PCR bead assay (BARCODE-MT). FIG. 1A shows a 96-well multi-well plate. FIG. 1B shows hybridization of PCR products to beads carrying target-specific nucleic acidprobes on 96-well plates. FIG. 1C represents a single well of a 96-well plate containing the seven member bead arrays used for detection of risk-stratifying translocations in pediatric acute lymphoblastic leukemia. FIG. 1D shows an example of an apparatus useful for carrying out the new assay. FIG. 1E represents an example of measured reporter fluorescence for a single patient with acute lymphoblastic leukemia carrying bcr/abl fusions of the b3a2 type (column “b3a2”).

FIGS. 2A to 2C are graphic results of the methods. FIG. 2A shows a representation of an electrophoresis gel and FIG. 2C shows a three-dimensional graph produced by a Luminex® 100 device generated by cell lines carrying chromosome translocations targeted by the acute lymphoblastic leukemia assay. FIG. 2B is a representation of standard Southern blot hybridization, which shows that the probes are highly specific over two to three logs of target concentration.

FIG. 3 is a three-dimensional graph that shows the sensitivity of the BARCODE-MT-ALL assay. The assay can reliably detect fusions even when the RNA accounts for as little as 1% of the input population.

FIG. 4 is a three-dimensional graph that shows the specificity of the BARCODE-MT-ALL assay in detecting risk-stratifying translocations in pediatric acute lymphoblastic leukemia.

FIG. 5A is a three-dimensional graph showing a comparison of BARCODE-MT-ALL with single-target PCR in the detection of risk-stratifying translocations in a cohort of sixty pediatric patients with acute lymphoblastic leukemia (ALL). The BARCODE-MT-ALL identified all translocations in perfect concordance with the single-target PCR results indicating 98% accuracy. FIG. 5B is a PCR assay corresponding to each of the sixty samples.

FIG. 6 is a table of primers, probes, and reverse transcriptase oligonucleotide decamers used in the formulation of the acute lymphoblastic leukemia assay.

FIG. 7 is a table of the oligonucleotides used in the formulation of BARCODE-MT-HPV (human papillomavirus) in an assay used to determine with which of 45 different human papillomavirus subtypes 96 different patients were infected. The first and third columns list the oligonucleotides used for PCR amplification of 45 HPV subtypes. The second column lists the oligonucleotides attached to the beads for hybridization to target amplified HPV nucleic acid.

FIG. 8 is a 45×45 matrix showing 98% specificity of BARCODE-MT-HPV probes in the detection of HPV viruses. The BARCODE-MT-HPV assay compares favorably with current methods such as Digene Hybrid Capture II®.

### DETAILED DESCRIPTION

The invention is based on the use of fast high-throughput assays to reveal specific genetic differences useful in the optimization of medical diagnosis, prognosis and therapy (risk-adapted therapy). Genetic differences can include DNA mutations, chromosomal rearrangements, gene copy number fluctuations, gene expression profiles (e.g., signatures), polymorphisms, and the presence of viral or bacterial nucleic acid, which can predispose an individual to a disease such as cancer (e.g., human papilloma virus (HPV) as a common cause of cervical carcinoma or anogenital cancers).

The invention provides methods to rapidly perform “targeted gene or transcript (RNA) profiling” in cancer that exploits the versatility of bead microarrays to be configured efficiently, rapidly and cost effectively in a myriad of different targeted profiling assays. The methods employ available technology (e.g., Luminex® xMAP beads and Luminex 100® instrumentation; Becton Dickinson Cytometric Bead Array (CBA) and Becton Dickinson FACSCaliber® instrumentation). Targeted gene sequences are obtained from public databases. Expression signatures are obtained from public cDNA databases and from hundreds of published massive parallel expression profiling assays in a variety of cancers PubMed). In most instances, a limited number of transcripts, usually well below 100, are sufficiently informative to produce a “signature.” Databases of gene sequences or individual signature sequences are searched and oligonucleotide probes synthesized to target only informative nucleic acid needed to generate strongly predictive algorithms. The oligonucleotide probes can then be attached to beads using standard chemistry. The targeted bead microarray is then hybridized with cRNA or DNA or amplified target nucleic acid from diseased tissue (e.g., tumors) or transformed cell lines, and control tissue or cells lines and read on a fluorescence detecting instrument such as the Luminex100® instrument. An Excel algorithm is used to normalize and display the data and to determine the presence, absence, or relative levels of a given signature on the tested tumor or cell lines. Bead microarrays have been used in transcriptional profiling of Arabidopsis (Yang et al., Genome Research 11: 1888-1898, (2001)). However, such bead microarrays have not been applied to transcriptional profiling of cancer, which is predominantly done with solid phase (chip) microarrays.

The invention takes advantage of the fluid phase nature of arrayed beads, which allows more expedient testing of only about six hours from sample collection to diagnosis. Additionally, use of the bead microarray technology is cost effective relative to the solid phase assays currently employed for transcriptional profiling. The BARCODE-MT assay allows the simultaneous detection of approximately 100 different nucleic acid targets in a single reaction vessel and read-out cycle. Transcriptional profiling promises to revolutionize the approach to cancer diagnosis and therapy. In its current discovery form, it is very costly, complicated, and not readily applicable to the cancer patient. Because of the usefulness of transcriptional profiling, there has been great pressure by scientists and from the general public to institute transcriptional profiling in the clinic. Bead microarrays for this purpose, as described herein, as an attractive alternative to solid phase microarray, could potentially become the dominant technology in the clinical setting.

The invention simplifies genetic risk-assessment in a variety of disorders, including acute lymphoblastic leukemia (ALL) in children and in adults. For example, the invention provides an assay for four established risk stratifying lesions and their variants for ALL within six hours of sample procurement. The new assays can be easily extended to include other targets such as point mutations, tandem gene duplications, deletions, or the detection of viral or bacterial nucleic acid for various disorders that may prove prognostically important in the coming years, such as Flt-3 mutations in acute myeloid leukemia in adults.

The new BARCODE-MT assay provides fast and accurate analysis of clinical samples by multiplexing target testing at both the amplification and detection steps. For multiplex detection, the invention uses bead microarrays because of their attractive cost/sample ratio and the flexibility of the format for devising new tests or modifying existing ones. The use of bead microarrays solves the conundrum of multiplex PCR, i.e., the increasing difficulty in unambiguously identifying a specifically amplified target by size alone (e.g., by gel electrophoresis or capillary electrophoresis) as the number of possible targets increases. Because bead microarrays are customized and compatible with fast flow read-through systems, fluid phase bead microarrays compare favorably with solid phase arrays for these types of assays. The adoption of a high-speed hybridization-based detection method is also amenable to automation, which is an important feature, because automation reduces operator input and the associated risk of error. Because of the favorable signal-to-noise ratio of our assay, case calling is unambiguous as demonstrated by the mixing and coding experiments described below, as well as the perfect correlation between single target PCR and BARCODE-MT.

The new assay is superior to conventional cytogenetics because, in addition to detecting lesions such as t(12;21), which are not easily identified cytogenetically, the new assay identifies additional translocations missed by conventional cytogenetics. Besides low performance proficiency, there are a number of situations that may lead to the failure of karyotyping in identifying a relevant translocation. Among these the most important are the presence of cytogenetically complex translocations that cannot be easily identified as one of the prognostically relevant translocations, the presence of a risk-stratifying fusion transcript in the absence of karyotypically evident changes and, the occurrence of mimics, that is, translocations with breakpoints in the vicinity of bona fide prognostically relevant translocations that do not involve the relevant genes. None of these issues affects the results obtained by BARCODE-MT, because this assay detects the pathogenically relevant hybrid mRNA message or exogenous DNA such as that from a virus or bacteria.

Another significant feature of the new assay is the short turn-around time. The new assay can be accomplished in as little as six hours. Short turn-around time is essential when the result of a test is required for an emergent therapeutic decision, such as is the case in providing therapy for children with acute leukemia. Another important feature of the assay is its high throughput capacity. Given that the bead microarray used in the experiments described below was seven members deep and that the analysis time for a set of 58 samples was approximately 30 minutes, the maximum theoretical throughput of the assay is about 5,040 targets, or 720 patient samples per work day. This calculation assumes six hours of instrument data collection time, seven translocation targets per assay tube, and limitless human and PCR laboratory resources. These values are several orders of magnitude greater than throughput values that can be achieved with other hybridization-based methods. These characteristics make the new BARCODE-MT assay equally well suited for tertiary care facilities and centralized national referral laboratories such as those of the Childrens Oncology Group.

General Methodology

In general, the new methods include obtaining appropriate probes that hybridize or bind to specific target genetic lesions that correspond to a specific disorder (e.g., risk-stratifying translocations), amplifying the mRNA for the targets using RT-PCR or DNA (genomic, mitochondrial or viral) PCR, and identifying the amplified PCR products by solution hybridization. In the case of identifying human papillomavirus (HPV) in a subject at risk for carcinoma of the cervix, the viral DNA is amplified by PCR and identified by solution hybridization to bead arrays. Solution hybridization can be done using a fluid bead array assay by constructing oligonucleotide-bound beads, exposing the beads and probes to the labeled PCR products under hybridizing or binding conditions, and then analyzing the combined sample/beads by detecting the level of fluorescence (e.g., by flow cytometry). Flow cytometric measurements or other means of measuring and detecting fluorescence are used to classify the beads within a set of beads and to determine the presence or absence of a particular target sequence within a test sample.

For example, oligonucleotides from a region of a genetic lesion are synthesized and coupled to a microsphere (bead) by standard techniques such as by carbodiimide coupling. To perform a test in accordance with the invention, a nucleic acid, e.g., DNA or RNA, is subjected to PCR amplification using standard techniques including primers necessary to amplify the particular region of DNA. The PCR product is then incubated with the beads under conditions sufficient to allow hybridization between the amplified DNA and the oligonucleotides present on the beads. One of the primers used in the PCR, the one priming synthesis of the DNA strand complementary to the probe sequence, incorporates a fluorescent molecule, or a non-fluorescent molecule such as biotin that can be used to attach fluorophores after PCR, rendering the PCR product fluorescent when reacted with fluorophore-labeled streptavidin. Other labels can be used as well. Aliquots of the reacted beads are then run through a dedicated flow cytometer, and the intensity of fluorescence on each bead is measured to detect the level of fluorescence, which indicates the presence, absence or relative level of targets in a sample or the quantity of target. The beads are addressable and are identified by bead-specific fluorescence (at wavelengths different from those used for the analyte) as well. This characteristic is useful since each set of beads may be bound to a different set of oligonucleotides and thus can be easily differentiated from each other. By no means, however, is the proposed assays or dependent on XMAP beads (Luminex) to classify the array, and alternative classification technology could be equally suitable. These include microtransponder-based beads, quantum dot-encoded beads, Raman-active nanoparticles, resonance light-scattering beads, photoactivated silver nanodot fluorescence beads, etc. These general methods, and devices that can be used to carry out the new assays, are described in U.S. Pat. Nos. 5,736,330; 5,981,801; 6,046,807; and 6,057,107; which are all assigned to Luminex® Corporation (Austin, Tex.).

In some instances, due to effective competition of target sites by target complementary strand, the kinetics of capture probe/target hybridization with conventional double strand DNA (dsDNA) PCR products, rendered single-stranded by heat or alkali denaturation, may become quite unfavorable. Thus, standard PCR methods can lead to low signal amplitude and low signal-to-noise ratios. In this situation it may be necessary to render targeted PCR products single stranded by means that lead to an excess of the probe target strand, thus eliminating probe/complementary DNA strand competition. Most available methods, such as asymmetric PCR and lambda exonuclease digestion are difficult to optimize for multiplex PCR.

Another technique to address low signal-to-noise ratio is to use Lambda exonuclease, which digests the phosphorylated strand of dsDNA, to render PCR products single stranded. If one of the primers used for PCR is phosphorylated, hemiphosphorylated dsDNA product will be produced, which upon Lambda exonuclease digestion will become single stranded. However, these methods suffer from the inability of the exonuclease to digest across certain dsDNA regions, such as dsDNA that is not completely and accurately matched, making this technique unpredictable. One way solved this problem is by using a phosphorothioate-linked primer. Phosphorothioate linked primers are resistant to digestion by some nucleases allowing one to protect one strand of the DNA to the digestive actions of some nucleases. A more suitable and convenient way of solving this problem is by performing what is described herein as LogLinear PCR. See Examples, infra.

In LogLinear PCR, conventional PCR (logarithmic PCR) is followed by Exonuclease I (Exo I) treatment to remove single-stranded primers, heat inactivation of Exo I, addition of a single primer, and performing a second PCR reaction (linear PCR). Because only a single primer is included in the second PCR reaction (linear PCR), abundant single stranded DNA product will be generated. This technique generates abundant ssDNA greatly improving PCR product hybridization to the bead microarray. Without the need for optimization and the problem of unpredictability, this technique leads to a large increase in signal intensity (20-50 fold) and significantly improves the signal-to-noise ratio because ssDNA target has been made more abundant. Forms of amplification other than conventional FCR can be used for the first step.

The fluorescent oligonucleotide probes can be prepared by standard methods, such as those described in U.S. Pat. No. 5,403,711.

The reacted sample is analyzed by detecting the level of fluorescence (e.g., by flow cytometry, e.g., using a Luminex® 100 apparatus or other suitable instrument such as standard flow cytometers (Coulter, Beckton Dikinson, etc.), to determine a change in the fluorescence intensity of the beads. The results can be analyzed using standard methods. The level of fluorescence on a particular bead is proportional to the amount of target (i.e., single-stranded PCR product) in the sample hybridized to its cognate probe.

After subtraction of background fluorescence, the results are expressed as light units relative to a control sample containing, or lacking, appropriate targets. Thus, large signal differences in fluorescence between samples containing or not containing relevant targets are produced leading to unambiguous sample scoring.

Transcriptional Profiling

In one embodiment of the genetic risk-stratifying method “gene expression signatures” (GES) diagnostic of a neoplasm, or defining a certain prognosis, or indicating particular resistance or susceptibility to certain medical therapies are detected. The selection of informative RNA transcripts can be achieved by mining public domain databases containing raw data of large scale mRNA profiling expeditions in cancer. The raw data can be subjected to a number of statistical algorithms to obtain transcripts that discriminate between a number of different biological characteristics important for the clinical management of patients with cancer. Alternatively, entire gene pathways known to be involved in certain cancers can be profiled by the design, a priori, of bead microarrays containing probes for all relevant gene transcripts in the pathway.

There are many available websites in the public domain which contain the expression profile data of many different cancers and tumor types. These databases contain a number of gene transcripts, usually well below one hundred, that allow the discrimination among groups of tumors with different biological and clinical behavior and varying responses to therapy. Most of the research which has supplied the information for these databases has been funded by the federal government. Some of these databases can be found on the internet under the addresses:

research.dfci.harvard.edu/korsmeyer/Over_under.htm

—genome.wi.mit.edu/mpr/lymphoma/

—genome.wi.mit.edu/mpr/CNS/

nature.com/cgi-taf/DynaPage.taf?file=/nature/journal/v403/n6769/full/403503a0_fs.html

nature.com/cgi-taf/DynaPage.taf?file=/nature/journal/v406/n6795/full/406532A0_fs.html

ncbi.nlm.nih.gov/geo/

www.dnachip.org/

young39.wi.mit.edu/chipdb_public/

genome-www.stanford.edu/nci60/

industry.ebi.ac.uk/˜alan/MicroArray/

hgmp.mrc.ac.uk/GenomeWeb/nuc-genexp.html

discover.nci.nih.gov

Genomic Profiling

Bead microarrays can be designed to contain gene dosage probes of genes known to be deleted or amplified in cancer and that convey diagnostic, prognostic, and therapeutic information. Gene dosage probes are probes against a gene of interest and probes against a control gene which when used together can determine levels (e.g., dosage, e.g., number of copies) of a gene relative to control. For example, increased copies of a gene is important in some cancers to determine proper treatment and prognosis. In the case of breast cancer, for example, a currently used treatment, a monoclonal antibody against the protein encoded by Her2/Neu, is only efficacious if the patient has an increased number of Her2/Neu gene copies. If the patient does not have an increased number of gene copies, the therapy is not efficacious. These probes are also useful in determining if a gene deletion or amplification is associated with a certain cancer. Gene rearrangement probes can be used to determine if a cancer is associated with certain gene rearrangements. For instance, the c-myc gene is frequently rearranged in Burkitt lympoma. The cyclin D1 gene is frequently rearranged in mantle cell lymphoma; the Bcl-2 and Bcl-6 genes are frequently rearranged in follicular lymphoma; and, the Bcl-10 gene is frequently rearranged in marginal cell lymphoma.

Applications

BARCODE-MT is also suitable for the investigation of other genetic lesions in leukemia such as point mutations, tandem duplications, and deletions. For example, the new assay can be used to detect these lesions in the Flt3 receptor gene, which occur in a high proportion of adults with acute myelogenous leukemia (AML).

Another potential application of BARCODE-MT -based assay is in targeted genomic or transcriptional profiling of cancer (e.g., acute myelogenous leukemia, chronic myeloproliferative disorders, chronic lymphoproliferative disorders, lymphomas (Hodgkin's and non-Hodgkins), lung cancer, prostate cancer, breast cancer, cervical cancer, anogenital cancer, and colon cancer). Transcriptome-wide expression profiling using solid-phase microarrays is a powerful technique to uncover gene expression patterns associated with certain characteristics of a tumor including histogenetic origin, clinical and biological features, and prognosis. Transcriptome-wide screens however are complex and expensive and at this time cannot be used in the routine care of patients with cancer. BARCODE-MT can be easily adapted to expression profiling of up to 100 genes at a time making it ideal for use in targeted transcriptional profiling screens uncovered by the more complex and extensive solid phase array screens used in the research arena.

The new assays simplify and improve genetic testing for currently known risk-stratifying genetic lesions, e.g., in pediatric acute lymphoblastic leukemia. The assay platform can be easily adapted for testing in adults with acute leukemia and is sufficiently flexible to be easily adapted to transcriptional profiling and a number of other nucleic and protein assays useful in the management of patients with hematological and non-hematological cancers. Examples of other disorders that can be assessed include: acute myelogenous leukemia, chronic myeloproliferative disorders, chronic lymphoproliferative disorders, lymphomas (Hodgkin's and non-Hodgkins), lung cancer, prostate cancer, breast cancer, cervical cancer, anogenital cancer, colon cancer and conditions that may predispose to cancer such as viruses and genetic polymorphisms.

The same type of assay can be used for a variety of related purposes such as the following: (1) risk-stratifying lesions other than translocations, (2) targeted transcriptional profiling of hematological and non-hematological cancers for the purpose of diagnosis, risk-assessment, or response to diverse therapies, (3) multiplexed targeted single nucleotide polymorphism of hematological and non-hematological cancers for the purpose of risk stratification or response to therapy, and (4) rapid multigene mutational screening in hematological and non-hematological cancers for the purpose of diagnosis, risk-assessment, or therapy.

The assay is also useful in screening for risk factors for the development of cancer such as papillomavirus infection in cervical carcinoma in women. Various strains of human papillomavirus confer different levels of risk for cancer development. This application involves the use of DNA sampling from the subject rather than RNA, as is the case in transcriptional profiling.

HPV is the common etiologic agent of carcinoma of the uterine cervix and anogenital cancers. The prevalence of HPV infection in women is high (20-30%). In spite of the high incidence of infection, however, cervical carcinoma develops in only one in every thousand infected women, making screening for the virus itself insensitive and not cost-effective. However small, it is clear that the risk of cancer development depends on both viral and host factors. The latter remain poorly defined and poorly understood while the former, the virus, is known to predispose to cancer in a subtype-dependent manner. Currently, mucosal HPVs are classified as low-risk, intermediate-risk, or high-risk according to their propensity to induce cancer. In most countries, including the United States, the Pap test is the primary means of screening for carcinoma of the cervix. The Pap test is a cytology test that assesses cells from the uterine cervix for changes associated with tumor development. Approximately 6-8% of these Pap smears result in ASCUS (abnormal squamous cells of unknown significance), an ambiguous result meaning that the pathologist reading the assay cannot determine with certainty whether there is enough evidence to warrant further invasive studies. The recently published 2001 Consensus Guidelines for the Management of Women with Cervical Cytological Abnormalities (Wright et al., JAMA 287:2120-2129 (2002)) recommends that HPV testing be performed in all women with an ASCUS result on a Pap test. High-risk HPV positive women are then referred for colposcopy (a more invasive test in which biopsies of the uterine cervix are taken under direct visual guidance), while those who test positive for low-risk HPV types or test negative for HPV are followed with annual repeat Pap tests. It is therefore important to identify with certainty whether a woman with a Pap test result of ASCUS (an estimated 1,800,000 in the United States) carries HPV and if she does, which type of HPV she carries.

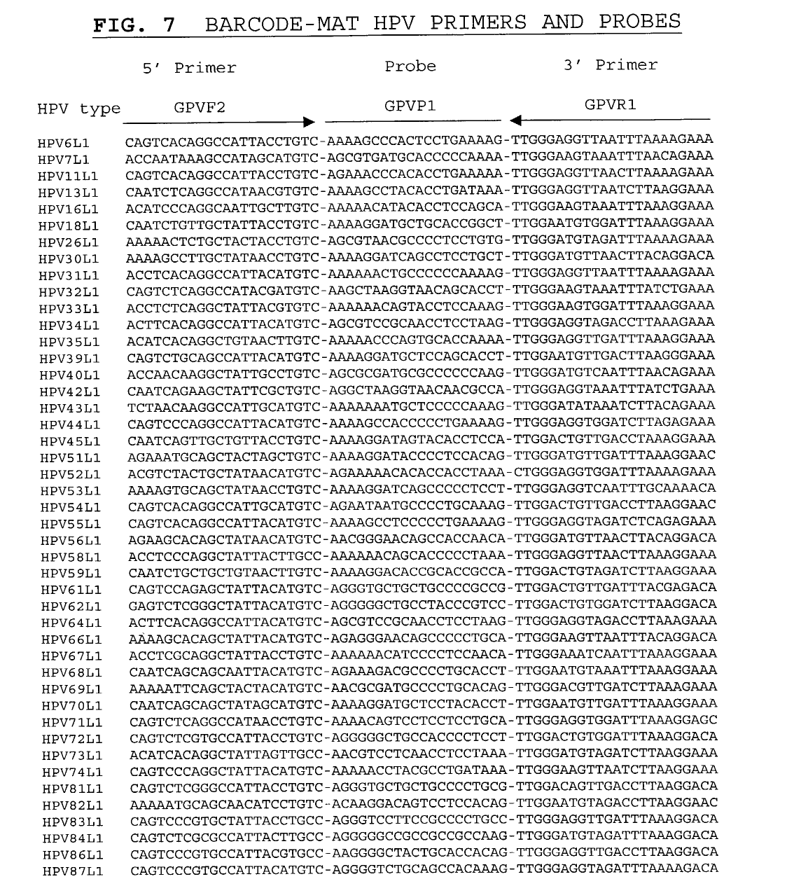

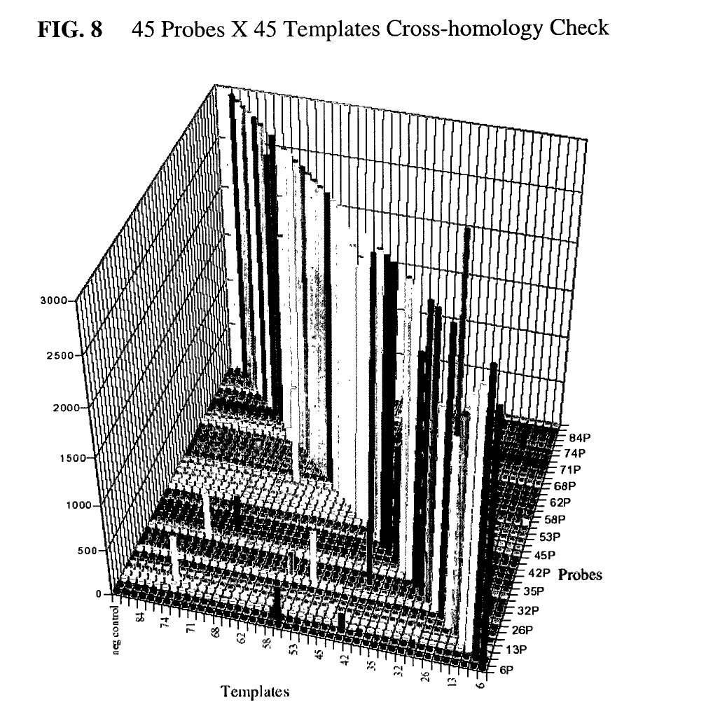

Commercial tests for HPV detection/typing are currently on the market, such as Digene's Hybrid Capture 11® and Ventana's Inform HPV. The new methods described herein, overcome limitations of these known tests. The new methods can use standard PCR as an amplification method. PCR is widely regarded as the gold-standard in HPV detection. It also uses linear PCR as a means of improving results by providing a greater number of single stranded PCR product for hybridization to beads, thus improving signal to noise ratio. Bead microarrays are then used as the readout technology. The new methods described herein are very sensitive and specific, and can type 45 individual HPV types and mixed-infections (more than one HPV type) in a single tube PCR/readout cycle. See Example 2 and FIG. 7 and FIG. 8. The new methods are relatively operator independent and can be fully automated, and are thus fast, accurate, and can be used in high throughput format.

Optimizing Therapy Based on Assay Results

The object of the new assays is to determine whether a sample contains one or more specific genetic lesions, and in particular, lesions that have prognostic value. In the specific case of acute lymphoblastic leukemia, there are known therapies indicated for specific genetic lesions, e.g., risk-adapted therapies. See, e.g., Ferrando et al., Semin. Hematol., 37:381-95 (2000).

Specifically, if the sample indicates a BCR/ABL fusion, the prognosis is poor, and the patient may require allogeneic bone marrow transplant upon initial remission. If the sample indicates an ALL1/AF4 gene fusion or a E2A/PBX fusion, the prognosis is also poor, and the patient must receive initial intensive therapy. These patients can do well if given appropriate early intensified therapy. For the ALL1/AF4 gene fusion, for example, intrathecal prophylactic therapy may also be required. On the other hand, if the sample indicates a TEL/AML1 fusion, the prognosis is fairly good, and the patient can be treated with standard chemotherapeutic regimens. In another example, by knowing what specific subtype of HPV a patient is infected with can help determine what risk category for cancer that patient is in. In general, it can be important to know whether the patient has a particular DNA translocation before therapy is administered.

### EXAMPLES

The invention is further described in the following examples, which do not limit the scope of the invention described in the claims.

Example 1

### Leukemia Assay in Cell Lines and Patients Samples

Leukemia cell lines carrying the risk-stratifying lesions assayed in this study were grown in RPMI 1640 supplemented with 20% fetal bovine serum, antibiotics, and glutamine.

The following cell lines were used as positive controls for multiplex RT-PCR reactions:

K562 for CML-type BCR/ABL^{P210}, SupB5 for ALL-type BCR/ABLP¹⁹⁰, RS4; 11 for ALL1/AF4, 697 for E2A/PBX, and REH for TEL/AML1. SUD-HL6, a follicle center cell lymphoma cell line, was used as a source of RNA devoid of a fusion transcript (negative control). To determine the sensitivity of assays, cell mixing experiments were carried out by diluting cells carrying the relevant genetic lesion into control cells prior to extraction of RNA or by mixing known amounts of RNA from cell lines bearing translocations with SUD-HL6 or normal bone marrow RNA.

In most experiments, we used RNA mixing rather than cell mixing since RNA mixing is simpler, more reproducible, quantitative, and hence more accurate. To determine the sensitivity of the assay in a clinically relevant manner, cells from patients diagnosed with acute lymphoblastic leukemia carrying TEL/AML or BCR/ABL transcripts were serially diluted into normal peripheral blood samples prior to cell isolation and RNA extraction. Use of clinical samples avoids overrepresentation of target RNA due to the well-known overabundance of RNA hybrid transcripts in leukemia cells grown in culture as compared with bone marrow cells.

Samples from sixty-two cases of acute lymphoblastic leukemia in patients 18 years of age or younger, which had been cryopreserved in our laboratory were also studied without prior knowledge of cytogenetic or clinical features. RNA was extracted from cell lines and leukemia samples were cryopreserved with Trizol (Gibco/BRL) following the manufacturer's instructions. RNA was quantified by spectrophotometry.

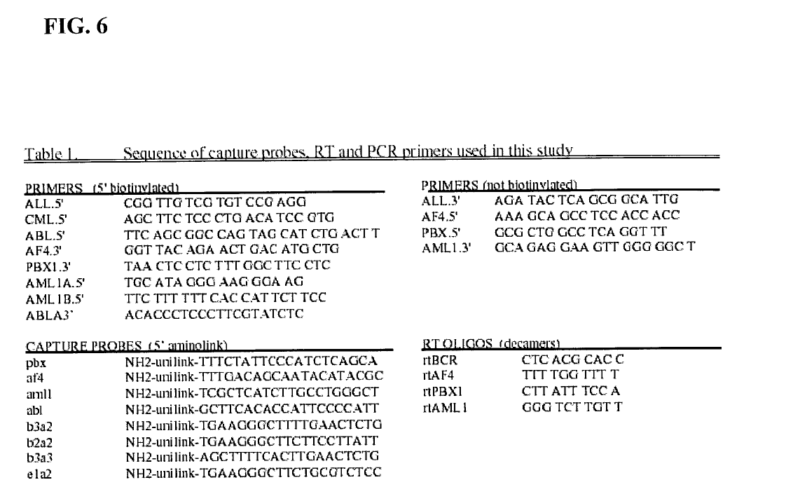

Four translocations occurring in pediatric acute lymphoblastic leukemia, with well-established risk-stratifying value, were the target of this study. These are the t(9;22)(q34.1;q 1) leading to BCR/ABL fusion, t(4;11)(q21;q23) leading to ALL1/AF4 gene fusion, t(1;9)(q23;p13) leading to E2A/PBX fusion, and t(12;21)(p13;q22) resulting in TEL/AML1 fusion. See, e.g., Ferrando et al., Semin. Hematol., 37:381-95 (2000). The gene fusion mRNA for all four translocations and their variants was amplified on a single RT-PCR reaction using standard techniques. For most reactions 0.5 μg of total RNA was reverse transcribed in 21 μl for 0.5 hour at 37° C. using 120 U of MMLV Reverse Transcriptase (Gibco/BRL) a mixture of decamers specific for each RNA fusion (see the table in FIG. 6) and conditions specified by the enzyme manufacturer. A 5 μl aliquot of cDNA was amplified by PCR on a PerkinElmer 9600 thermal cycler using polypropylene, V-bottom, 96-well plates (Multiplate 96, MJ Research, Waltham, Mass.) and AmpliTaq polymerase (Perkin Elmer). Primers and cycling conditions were essentially as described in Scurto et al. (Leukemia, 12:1994-2005, 1998) with some modifications (FIG. 6). Each 25 μl reaction contained a mixture of fusion specific 5′ and 3′ primers, 80 mM dNTPS, 15 mM Tris pH 7.8, 3.5 mM MgCl₂ 5% DMSO, and 15% glycerol. One of the primers in each pair carried a 5′ biotin group. After an initial denaturation step of 2 minutes at 95° C., 13 cycles of 94° C. for 30 minutes, 63° C. for 1 minute, and 72° C. for 1 minute were carried out, decreasing the annealing temperature by 1° C. per cycle. This was followed by 35 cycles identical to the 13Th cycle. The last extension step lasted 7 minutes. Reactions were then brought to 4° C. RNA samples devoid of any of the translocations amplified by the assay, as well as reactions containing no RNA, were routinely included as negative and carry-over contamination controls, respectively.

The production of ssDNA target was facilitated by the addition of 2U per reaction Exonuclease I (New England BioLabs, Beverly, Mass. (Exo I)) to the terminated PCR reaction. After inactivating Exo I by heating, only one of the primers, the one priming synthesis of the target strand complementary to the probe, was then added to the mixture and a round of linear PCR performed to produce the final target (or probe) mix. This simple procedure which can be used in any application requiring the production of ssDNA targets or probes involves the complete digestion of the initial ssDNA primer pair at the completion of the logarithmic amplification phase followed by the addition of one of the two primers and a phase of linear polymerase amplification. The product of this reaction can then be used, without further modification as the target (or probe) in the hybridization reaction. This simple linear PCR after logarithmic amplification protocol (referred to herein as LogLinear PCR) has the advantages of low signal-to-noise ratio, higher signal, ease of optimization even for multiplex PCR, and the ability of Exo I, to effectively deplete the PCR reaction of the oligonucleotide priming synthesis of the non-target sequence making the use of products of this procedure more predictable.

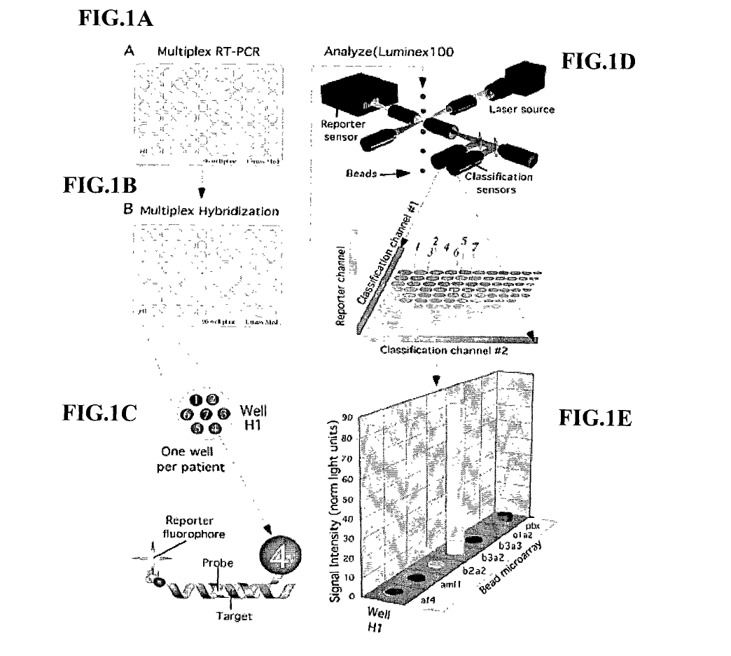

The identity of amplified PCR products was determined by solution hybridization with target-specific capture oligonucleotides covalently attached to beads, e.g., LabMAP T beads (Luminex Corp., Austin, Tex.). For each analyte, 5,000 beads, e.g., LabMAP3 beads, were used and information was collected from 100 of these 5 μm polystyrene beads. Each analyte was represented by one set of beads. Each bead set is encoded with varying amounts of two spectrally-distinguishable red fluorescent dyes (classification dyes) (FIG ID). Each bead set can be coupled to a probe oligonucleotide (FIG. 1B) and, after batch reaction with the test sample, unambiguously identified in a high throughput analyzer (FIG. 1C), which also measures bead-associated reporter fluorescence (reporter dye) (Luminex 10®, Luminex Corporation, Austin, Tex., FIGS. 1A-D).

Each bead array was formulated to contain seven bead sets of 5,000 beads each for a total of 35,000 beads per assay. Information was collected until all seven bead sets in the array had accrued a minimum of 100 events. This translocation assay, resulted in a total assay read-through time of less than 30 seconds per sample (one patient per well of 96-well microtiter plate). Each bead was coupled to capture/probe oligonucleotides using carboimidine, a heterobifunctional crosslinking reagent that reacts with both the amino group present on the 5′ end of each capture probe oligonucleotide and the carboxyl group present of the bead surface leading to covalent coupling (FIG. 1B). Capture/probe oligonucleotides are listed in the table in FIG. 6. After coupling bead concentration was adjusted to 10⁶ ml⁻¹ and equal aliquots of each bead were combined onto a master bead array mix (FIGS. 1A-B).

Hybridizations were carried out in 2 M tetramethyl-ammonium chloride (TMA), conditions in which the melting temperature of the oligonucleotides is solely dependent on chain length (and independent of base content and context). This permitted the use of dodecamers (FIG. 6) as capture oligonucleotides in a single tube hybridization reaction at a single temperature for all target PCR product/probe combinations. Hybridizations were initiated by transferring, with a multi-channel pipette, aliquots of denatured (kept at 99° C. on a 96-well thermal block) terminated PCR reactions into a 96-well multiplate containing aliquots of bead arrays suspended in 1.5×TMAC (1×=2M TMA, 0.1% sarcosyl, 50 mM Tris, 4 mM EDTA).

Hybridizations were carried out for 30 minutes at 54° C., after which samples were washed in 1×TMA, spun down, and resuspended in 1×TMA containing 1.0 μg/ml of streptavidin-phycoerythrin (Molecular Probes, OR), incubated for 5 minutes at 45° C., spun down, and resuspended in 1×TMA. Samples were then automatically injected into the Luminex 100® analyzer using a XY platform. The Luminex 100® instrument is similar in concept to a flow cytometer and utilizes two laser light sources, one to excite the two classification fluorophores, the other the reporter fluorophore (FIGS. 1C and 1D). Numerical results were expressed as mode light intensity units normalized to correct for background fluorescence and the varying hybridization efficiencies of the different target/capture probe combinations. Data was plotted using standard techniques and standard software (Microsoft® Excel®).

Using dextran separation and Trizol RNA extraction, PCR ready cDNA could be obtained from whole blood or bone marrow specimens in less than two hours. Optimization of a previously published multiplex amplification protocol (Scurto et al., 1998) led us to PCR conditions that accomplished amplification in approximately 2 hours. LogLinear PCR, as described above, was then performed to facilitate the production of ssDNA target.

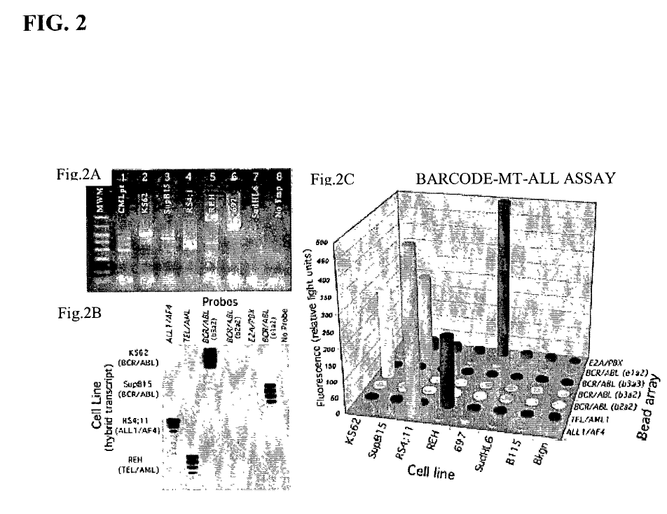

The bead microarray was hybridized to denatured PCR products for 30 minutes. The entire procedure from “sample arrival” in the laboratory to analysis in the Luminex 100® instrument could be carried out in 6 hours, well within the scope of a full work-day in a clinical laboratory. FIGS. 2A-C depict target specific amplification by multiplex RT-PCR (FIG. 2A), and the specificity of the capture oligonucleotide probes under the hybridization conditions developed for the BARCODED-MT assay (FIG. 2B). Note that there is virtually no cross-hybridization of probes to non-cognate target DNA under these conditions (FIG. 2B).

After subtraction of bead-specific background fluorescence using readouts produced by hybridization of the bead microarray to PCR reactions templated by translocation negative cDNA, the signal-to-noise ratio for five independent hybridizations, indicated by the five peaks in fluorescence, was greater than 40 for all targets (FIG. 2C).

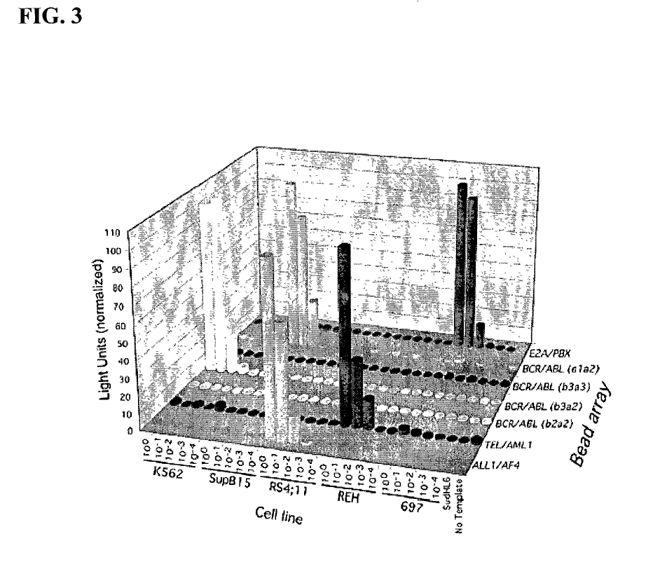

To determine the sensitivity of the assay we prepared RNA samples containing decreasing quantities of target RNA by serial tenfold dilutions of target RNA into background (target negative) RNA. Analysis of these samples after reverse transcription indicated that all targets could be reliably identified when they accounted for 1.0 percent or more of the input RNA (FIG. 3). In FIG. 3, the x-axis shows 5 different cell lines with ten-fold dilutions (10₋₁, 10^{-2, 10}^{-3, 10}⁻⁴) of input (10⁰) the y-axis are the measured units (normalized light units); and the z-axis are the different genetic lesions detected. This level of sensitivity is more that adequate for the intended use of this particular assay with these named probes, which is as an ancillary test in the diagnosis and management of acute lymphoblastic leukemia in children.

To determine the specificity of the assay, fifty-two coded RNA samples, prepared from mixing equal amount of RNA from translocation carrying cells and cells devoid of such targets, were simultaneously assayed using the 96-well multi-plate format for reverse transcription, amplification, hybridization, and assay read-out. The operator did not have access to the code until after completion of the assay and the graphic display of the results.

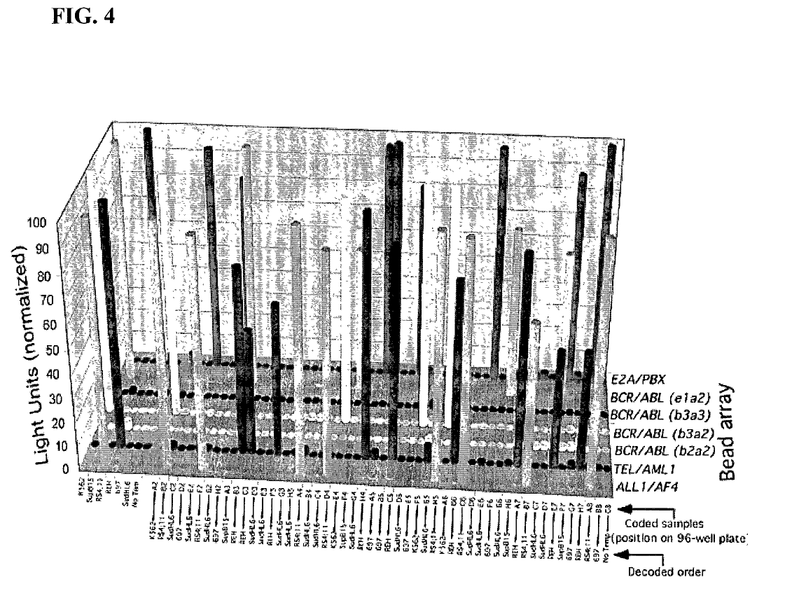

The assay was carried out in approximately 7 hours (starting from RNA). Automated instrument analysis time for 58 samples (50 unknowns, 5 positive controls, and 3 negative controls) was 25-30 minutes (˜30 seconds per sample) and occurred unattended. In this set of samples, BARCODE-MT-ALL correctly identified 4 of 4 CML-type BCR/ABL fusions, 4/4 ALL-type BCR/ABL fusions, 8 of 8 MLL/AF4 fusions, 8 of 8 E2A/PBX fusions, and 9 of 9 TEL/AML fusions. All 17 samples devoid of fusion transcripts scored negative on the assay (FIG. 4) indicating that the assay is highly specific (overall sensitivity and specificity of 100%) and suitable for clinical use.

Intra-assay and inter-assay reproducibility was high with intra- and inter-assay coefficients of variance of 4 to 8%. A hybridization temperature of 58° C. was optimal, but the useful temperature range was wide (+/−5° C., results not shown). This result is advantageous because it indicates that the new methods provide some flexibility during bead manipulation.

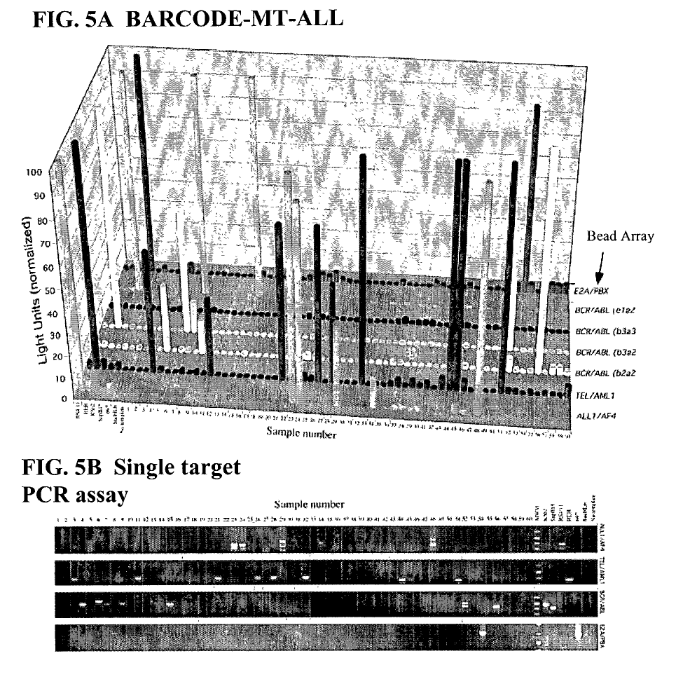

We next analyzed sixty samples from children with previously diagnosed acute lymphoblastic leukemia at our institution. RNA was prepared from cryopreserved samples and single-target RT-PCR was carried out as described above. Single-target RT-PCR is the current “gold standard” for diagnosis of ALL and other genetic disorders. These samples were studied without prior knowledge of the results of cytogenetic studies performed during diagnostic work-up. Analysis of these samples revealed a perfect correlation with the results of single target RT-PCR (FIG. 5). The new assay detected 8 of 8 TEL/AML translocations, 4 of 4 BCR/ABL translocation, and 3 of 3 MLL/AF4 translocations (FIG. 5A and confirmed by RT-PCR as illustrated in FIG. 5B). No E2A/PBX translocations were detected by BARCODE-MT-ALL or by single target RT-PCR. Forty-five samples were negative by both single target RT-PCR and BARCODE-MT-ALL. None of the TEL/AML translocations were detected cytogenetically, indicating that our assay, besides detecting translocations not usually visible on routine banded karyotypes, was also superior in detecting cytogenetically evident translocation (which may have been cytogenetically complex, or invisible).

### Example 2

### Human Papilloma Virus Screening

Pap tests which have had results of abnormal squamous cells of uncertain significance (ASCUS) are currently triaged by HPV testing. Patients infected with high-risk HPV types are subjected to colposcopy/biopsy, whereas those uninfected or infected with low-risk HPVs are followed with annual pap smears. Despite the proliferation of HPV assays none can accurately type all genital papillomaviruses in a simple high throughput assay.

A combination of multiplexed PCR and bead microarray read-out technology as described herein can be used to accurately type HPV in a simple high-throughput assay. Type-specific sequences of the L1 gene were obtained from GeneBank and aligned using clustalW. Starting with DNA extracted/separated from the Pap thin prep (Digene Corporation, Hybrid Capture II®) fluid, HPV-type specific regions of DNA were amplified using flanking consensus primer sites with sensitive Polymerase Chain Reaction (PCR)(for 5′ and 3′ PCR oligos refer to FIG. 7, first and third columns). Amplification primers were a previously published PGMY09 family and a newly designed GPVF family. See. FIG. 7. Type specific probes (GPVP) were generated from a sequence divergent area near PGMY09 and were tested for cross-homology by iterative sequence comparison (MacVector).

The production of ssDNA target was facilitated by LogLinear PCR. Basically, 2U per reaction Exonuclease I (New England BioLabs, Beverly, Mass. (Exo I)) was added to the terminated PCR reaction. After inactivating Exo I by heating, only one of the primers, the one priming synthesis of the target strand complementary to the probe, was then added to the mixture and a round of linear PCR performed to produce the final probe mix. This simple procedure which can be used in any application requiring the production of ssDNA targets or probes involves the complete digestion of the initial ssDNA primer pair at the completion of the logarithmic amplification phase followed by the addition of one of the two primers and a phase of linear polymerase amplification. The product of this reaction can then be used, without further modification as the target (or probe) in the hybridization reaction. This simple LogLinear PCR has the advantages of low signal-to-noise ratio, higher signal, ease of optimization even for multiplex PCR, and the ability of Exo I, to effectively deplete the PCR reaction of the oligonucleotides used in the log amplification step making the use of products of this procedure more predictable.

Single strand probes were attached to microarray beads with CDE chemistry (see FIG. 7, second column for a list of probes used). Array performance was evaluated by hybridizing the whole array with each individual target sequence in a 45×45 matrix, by including known types amplified from plasmid templates and by sequencing the target region of clinical samples for common and rare types. See FIG. 8. FIG. 8 is a 45×45 matrix showing that HPV type-specific probes recognize their type-specific target without false positives or false negatives. These results indicate that it is possible to type 45 viral types in a single tube. To test the assay in a clinically relevant manner, samples used consisted of known subtypes amplified from plasmid templates and clinical samples whose target region was sequenced for common and rare types. The BARCODE-MT-HPV compared favorably to current methods of HPV detection, such as the Digene Hybrid Capture II® test, since results obtained by BARCODE-MT-HPV were accurate and more informative. The x-axis and the y-axis represent each method. Equivalent assay results (e.g., the absence of false positives and false negatives by the new assays) were obtained for each method as seen by the row of bars down the center. The assay could accurately type 45 genital papillomavirus subtypes in 96 patients within 7 hours of sample collection, more rapid than current methods, and is suitable for both liquid cytology samples and conventional swab samples. The assay frequently detected mixed infections in clinical samples and correctly typed viral sequences obtained from plasmid templates and clinical samples. The BARCODE-MT-HPV assay compared favorably with Digene's Hybrid Capture II® and can be used for both clinical and epidemiological studies.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of simultaneously detecting the presence of multiple target nucleic acid molecules in a biological sample, the method comprising
(a) isolating and enriching target nucleic acid molecules from the biological sample;
(b) treating the enriched target nucleic acid molecules with Exonuclease I;
(c) performing linear PCR on the Exonuclease I treated enriched target nucleic acid molecule to produce linear PCR product; wherein only a single primer is used;
(d) obtaining beads coupled to an oligonucleotide molecule complementary to the amplified target nucleic acid molecules;
(e) forming a mixture by mixing the beads and the enriched linear PCR product nucleic acid;
(f) forming a reacted sample by incubating the mixture under conditions wherein if the enriched linear PCR product includes the target nucleic acid molecule, the enriched linear PCR product will hybridize to the oligonucleotide molecule;
(g) analyzing the reacted sample by determining the fluorescence of each bead analyzed; and
(h) detecting a level of fluorescence on the beads, wherein the level of fluorescence corresponds to a level of target nucleic acid molecule in the biological sample.

2. The method of claim 1, wherein the target nucleic acid comprises a genetic risk-stratifying lesion.

3. The method of claim 1, wherein the isolated nucleic acid comprises RNA or DNA.

4. The method of claim 1, wherein the enrichment is performed by RT-PCR.

5. The method of claim 1, wherein the enrichment is performed by PCR.

6. The method of claim 1, wherein the target nucleic acid comprises a risk-stratifying lesion in a gene associated with a cancer or a cancer predisposition.

7. The method of claim 1, wherein the method is used to optimize risk-adapted therapy for a disorder associated with the target nucleic acid.

8. The method of claim 7, wherein the disorder is leukemia, lymphoma, sarcoma, carcinoma, chronic myeloproliferative disorders, chronic lymphoproliferative disorders, lung cancer, prostate cancer, breast cancer, cervical cancer, anogenital cancer, and colon cancer.

9. The method of claim 8, wherein the leukemia is acute lymphoblastic leukemia or leukemia occurring in the adult population.

10. The method of claim 1, further comprising determining if a gene rearrangement is present or absent in the targeted nucleic acid molecule, wherein presence or absence of a gene rearrangement provides information to provide optimized diagnosis, prognosis and/or therapy.

11. The method of claim 1, further comprising determining gene dosage of the target nucleic acid molecule, wherein gene dosage provides information to provide optimized diagnosis, prognosis and/or therapy.

12. The method of claim 1, further comprising determining if the target nucleic acid molecule is mutated, wherein presence or absence of mutation provides information to provide optimized diagnosis, prognosis and/or therapy.

13. The method of claim 12, wherein the mutation is a gene fusion, a gene inversion, a gene deletion, or a gene insertion.

14. A method of simultaneously detecting the presence of multiple target nucleic acid molecules in a biological sample, the method comprising
(a) enriching isolated nucleic acid from the biological sample, wherein enrichment incorporates a detectable label onto a PCR product, wherein the PCR product may comprise a target nucleic acid;
(b) treating the amplified nucleic acid with Exonuclease I;
(c) performing linear PCR on the Exonuclease I treated amplified nucleic acid to produce linear PCR product; wherein only a single primer is used;
(d) obtaining addressable beads coupled to at least one oligonucleotide molecule complementary to the target nucleic acid;
(e) mixing the addressable beads and the linear PCR product to form a mixture;
(f) incubating the mixture under conditions allowing the linear PCR product to hybridize to oligonucleotide molecules that contain the target nucleic acid;
(g) analyzing the incubated mixture by determine the address of each bead analyzed by its fluorescence; and
(h) detecting a level of the detectable label on each of the addressable beads, wherein the level of the detectable label corresponds to a level of the target nucleic acid in the biological sample.

15. The method of claim 14, further comprising optionally removing unhybridized PCR product from the incubated mixture prior to analyzing the incubated mixture.

16. The method of claim 14, wherein the isolated nucleic acid consists of RNA or DNA.

17. The method of claim 16, wherein the isolated DNA comprises HPV DNA

18. The method of claim 14, wherein the target nucleic acid is a genetic risk-stratifying lesion.

19. The method of claim 14, wherein the amplification comprises performing RT-PCR.

20. The method of claim 14, wherein the amplification comprises performing PCR.

21. The method of claim 20, wherein the target nucleic acid is a risk-stratifying lesion in a gene associated with a cancer or a cancer predisposition.

22. The method of claim 21, wherein the cancer is selected from the group consisting of acute myelogenous leukemia, chronic myeloproliferative disorders, chronic lymphoproliferative disorders, lymphomas (Hodgkin's and non-Hodgkins), lung cancer, prostate cancer, breast cancer, cervical cancer, anogenital cancer, and colon cancer.

23. The method of claim 14, wherein the method optimizes risk-adapted therapy for a disorder associated with the target nucleic acid.

24. The method of claim 23, wherein the disorder is cancer or a cancer predisposition.

25. The method of claim 24, wherein the cancer is selected from the group consisting of leukemia, chronic myeloproliferative disorders, chronic lymphoproliferative disorders, lymphomas, sarcomas, carcinomas, lung cancer, prostate cancer, breast cancer, cervical cancer, anogenital cancer, and colon cancer.

26. The method of claim 25, wherein the leukemia is acute lymphoblastic leukemia or leukemia occurring in the adult population.

27. The method of claim 14, wherein the detectable label emits fluorescence.

28. The method of claim 14, wherein the detectable label is biotin.

29. The method of claim 14, wherein the method is used to optimize risk-adapted therapy for a disorder associated with a target nucleic acid.

30. The method of claim 29, wherein the disorder is leukemia, lymphoma, sarcoma, carcinoma, chronic myeloproliferative disorders, chronic lymphoproliferative disorders, lung cancer, prostate cancer, breast cancer, cervical cancer, anogenital cancer, and colon cancer.

31. The method of claim 14, further comprising determining if a gene rearrangement is present or absent in the targeted nucleic acid, wherein presence or absence of a gene rearrangement provides information to provide optimized diagnosis, prognosis and/or therapy.

32. The method of claim 14, further comprising determining gene dosage of the target gene, wherein gene dosage provides information to provide optimized diagnosis, prognosis and/or therapy.

33. The method of claim 14, further comprising determining if the target gene is mutated, wherein presence or absence of mutation provides information to provide optimized diagnosis, prognosis and/or therapy.

34. The method of claim 33, wherein the mutation is a gene fusion, a gene inversion, a gene deletion, or a gene insertion.
